(19) United States
(12) Patent Application Publication
(10) Pub. No.: US 2023/0302095 A1
(43) Pub. Date: Sep. 28, 2023

(54) INCREASING THE EFFICACY OF BIOLOGICAL THERAPEUTIC MOLECULES

(71) Applicant: Kyon Biotech AG, Zurich (CH)

(72) Inventors: Slobodan TEPIC, (US); Goran CVETKOVIC, (US)

(73) Assignee: Kyon Biotech AG, Zurich (CH)

(21) Appl. No.: 18/041,423

(22) PCT Filed: Aug. 13, 2021
(86) PCT No.: PCT/EP2021/072616

§ 371 Date: Feb. 13, 2023

(30) Foreign Application Priority Data
Aug. 14, 2020 (EP) 20191139.3

(51) Int. Cl. A61K 38/28; A61K 9/00

(52) U.S. Cl. CPC A61K 38/28; A61K 9/0019

(57) ABSTRACT

The present invention relates to improved delivery of therapeutic biologics with the molecular weight in the range from 10 kDa to 70 kDa or therapeutic nuclear acids with the molecular weight in the range from 6 kDa to 10 kDa by the concurrent deployment of an insulin-glucose clamp.

The present invention relates to medications, methods, and devices for increasing the efficacy of biological therapeutic agents. Particularly, the present invention relates to improved delivery of therapeutic biologics e.g. polypeptides with a molecular weight in the range from about 10 kDa to about 70 kDa or therapeutic nuclear acids with the molecular weight in the range from about 6 kDa to about 10 kDa by the concurrent deployment of an insulin-glucose clamp.

### BACKGROUND

Recent developments based on molecular genetics have led to many regulatory approvals for new biological therapeutic agents (biologics) including antibodies and antibody fragments as well as therapeutic nucleic acids. In vivo effectiveness of all of these compounds is however limited by their delivery to their targets which may be cells, bacteria, or viruses, typically outside of the vascular circulation.

Primary delivery into the blood circulation is the only viable method to get these drugs at least a chance of systemic mass transport, but ultimately, there are two fundamental physiological hurdles to overcome:
(1) Compounds having a molecular weight of 70 kDa or less are rapidly eliminated from the vascular circulation by glomerular filtration;
(2) Compounds having a molecular weight exceeding 70 kDa are mostly retained within the vascular circulation until elimination by various cellular components of the immune system.

Hence there is a conundrum facing most of the biologics: if larger than 70 kDa they are retained in the vascular system and thus prevented from reaching their targets; if smaller than 70 kDa they are rapidly eliminated via kidneys.

The molecular weight of antibodies, naturally occurring or engineered, is typically about 150 kDa; antibody fragments can be as small as 12 kDa (so-called nanobodies) and up to 50 kDa, or twice that size, well above the 70 kDa cutoff by kidney excretion, if linked to other fragments to prevent that path of elimination.

An approach to prolonging circulation half-life of smaller fragments is PEGylation (attaching chains of polyethylene glycol to the protein). That does not resolve the conundrum because pegylated molecules are definitely too large for an efficient extravasation.

Similar obstacles exist for therapeutic nuclear acids, which are typically in the range from 6 kDa to 10 kDa, still too large for mass transport by diffusion.

It is important to note in this context that the molecular weight of albumin, the main blood plasma protein, is 72 kDa and that under normal physiological conditions, only a small fraction of albumin leaks out into the interstitial fluid. When it does, it is readily detected as it leads to tissue edema. Albumin is found in the urine in traces only. Rate of glomerular filtration varies with the molecular weight—small molecules are returned to vascular circulation by specialized mechanisms of active transport.

Thus, it is an object of the present invention to overcome the above disadvantages and to provide means for increasing the efficacy of biologics.

### The Insulin-Glucose Clamp

We have found that the efficacy of biologics may be increased by assisting extravasation of these compounds, i.e. the transport from the vascular system into the interstitial fluid, by co-administering a biological therapeutic molecule together with an insulin and glucose. Particularly, the biological therapeutic agent may be administered by infusion, e.g. infusion over a period from several, e.g. at least about 5 or at least about 10 minutes to about 1 h or more, wherein a concurrent insulin-glucose clamp is provided, for example by administering, e.g. by infusion, insulin at a predetermined dose rate and administering glucose, e.g. by infusion, at a dose adjusted to maintain plasma glucose within an acceptable range.

Thus, a first aspect of the invention relates to an insulin for medical use wherein said insulin is co-administered with glucose and a biological therapeutic molecule.

A further aspect of the invention relates to a method for administering a biological therapeutic molecule to a subject in need thereof, wherein said biological therapeutic molecule is co-administered with an insulin and glucose.

According to the present invention, extravasation of the biological therapeutic molecule is assisted, e.g. by improving the transport of the biological therapeutic agent from the vascular system into the interstitial fluid system.

The present invention is for use in human medicine or in veterinary medicine. In certain embodiments, the biological therapeutic agent is administered together with insulin and glucose to a human. In certain embodiments, the biological therapeutic agent is administered together with insulin and glucose to a non-human mammal, e.g. a dog, a cat, a horse or cattle.

In particular embodiments, the insulin is co-administered with glucose as an insulin-glucose clamp. Accordingly, the insulin may be administered by infusion at a predetermined dose rate, e.g. at a predetermined constant dose rate. For humans, the insulin may be administered at a dose rate from about 1.5 to about 6 I.U./kg body weight/day, preferably from about 2 to about 4 I.U./kg body weight//day, more preferably about 3 I.U./kg body weight//day. For dogs, the insulin may be administered at a higher dose rate from about 3 to about 12 I.U./kg body weight//day, preferably from about 4 to about 8 I.U./kg body weight//day, more preferably about 6 I.U./kg body weight//day.

The insulin may be any type of natural or recombinant insulin or insulin analogue suitable for applying an insulin-glucose clamp. Preferably, the insulin is a rapid acting insulin or a short acting insulin, more preferably a rapid acting insulin. Examples of rapid acting insulin are insulin lispro, insulin aspart or insulin glulisine. Examples of short acting insulins are regular insulin or insulin velosulin.

According to the present invention, the insulin is co-administered with glucose. Preferably, glucose is administered by infusion. In this context, it is noted that the term “glucose” as used herein also includes a glucose containing oligosaccharide or polysaccharide capable of releasing glucose into the blood, for example, any type of dextrose, such as partial hydrolysis products from starch or maltodextrin. The administration of glucose may be adjusted to maintain the plasma glucose level within normal, physiological concentrations, e.g. from about 70 mg/dl to about 130 mg/dl. This may require delivery of about 10 g glucose/kg body weight/day for dogs at an insulin infusion dose rate of about 6 I.U./kg body weight/day; or delivery of about 5 g glucose/kg body weight/day for humans at an insulin infusion dose rate of about 3 I.U./kg body weight/day. This may be achieved by an infusion of a 10% glucose solution into a peripheral vein of an average person, started at a rate of about 150 ml/h for the middle dose rate of insulin (3 I.U./kg/day) and adjusted as needed. Dextrose, with an equivalent rate of infusion, can be used instead of glucose.

The delivery rate of a glucose solution can be controlled by a simple drip method from an infusion bag or bottle of e.g. 250, or 500 ml volume. Non-invasive monitoring of glucose can be performed by e.g. FreeStyle Libre from Abbott Laboratories.

Co-administration of insulin and glucose may start before, at or after administration of the biological therapeutic compound. Typically, co-administration of insulin and glucose is performed for at least about 3 h, at least about 6 h, for at least about 12 h or at least about 24 h and up to several days depending on the type and administration route of the biological therapeutic agent.

Administration of glucose may be accompanied by administration of a potassium salt such as KCI to compensate for potassium ions entering cells if a high dose of glucose is administered. Further, the treatment may be supported by concomitant administration of essential amino acids, electrolytes, fluids and/or antibiotics.

The biological therapeutic molecule may be a polypeptide, e.g. an antibody including a recombinant antibody or antibody fragment or derivative, an immunoglobulin fusion protein, an interferon, an interleukin or a cytokine, or a nucleic acid, e.g. a DNA, RNA or modified nucleic acid, i.e. a nucleic acid containing at least one modified building block.

The biological therapeutic agent can further be PEGylated or glycosylated, conjugated with another active agent or it can be modified in a different way.

In a particular embodiment, the biological therapeutic agent is an antibody including a complete antibody, e.g. IgG, IgM, IgA, IgD and IgE, an antibody derivative such as a single chain antibody, an antibody fragment and a conjugate of an antibody, e.g. a conjugate with a pharmaceutically active group such as a cytotoxin or a radioactive group.

In another particular embodiment, the biological therapeutic agent is an immunoglobulin fusion protein, e.g. a fusion protein of a cytokine or growth factor with a constant immunoglobulin domain and a conjugate of such an immunoglobulin fusion protein, e.g. a conjugate with a pharmaceutically active group such as a cytotoxin or a radioactive group.

In another particular embodiment, the biological therapeutic agent is a cytokine, an interleukin or an interferon including interferon-alpha, interferon-beta and interferon-gamma or a conjugate thereof.

In certain embodiments, the biological therapeutic agent is a compound having a molecular weight of more than about 70 kDa, e.g. a therapeutic antibody.

In certain embodiments, the biological therapeutic agent is a compound having a molecular weight of about 70 KDa or less, e.g. a molecular weight from about 4 kDa to about 70 kDa. For example, the biological therapeutic agent may be a polypeptide, e.g. a therapeutic antibody fragment, or another protein or peptide, which may have a molecular weight from 10 kDa to about 70 kDa, from about 5 kDa to about 70 kDa and particularly from about 10 kDa to about 50 kDa. Further, the biological therapeutic agent may be a therapeutic nuclear acid, e.g. an antisense oligonucleotide (ASO), an aptamer, or a therapeutic RNA (siRNA, microRNA or mRNA), which may have a molecular weight of about 4 kDa to about 20 kDa, particularly from about 6 kDa to about 10 kDa.

The biological therapeutic agent is administered to target the vascular system, e.g. by infusion or injection. In certain embodiments, the biological therapeutic agent is delivered to the subject by different means as the insulin and the glucose, e.g. by injection whereas insulin and glucose are administered by infusion, or by an infusion different from the insulin and glucose infusions. In certain embodiments, the biological therapeutic agent is administered by the same means as the insulin and/or the glucose, e.g. by co-infusion with insulin and/or glucose, particularly by co-infusion with insulin.

In a particular embodiment, the therapeutic agent, e.g. an antibody fragment is administered by infusion. Application of an insulin-glucose clamp as an adjuvant to the infusion of the biological therapeutic agent is typically of limited duration. After the infusion of the biological therapeutic agent is completed, the insulin-glucose clamp needs to be continued only to cover the first and perhaps second cycle of the systemic circulation. The molecules of the therapeutic agent, e.g. the antibody fragments are expected to return to the vascular system via lymphatic drainage, albeit after making their first round of attack on their specific targets.

Typically, the biological therapeutic agent is administered in a native, i.e. non-denatured form. Further, in certain embodiments, the biological therapeutic molecule is not an asparaginase or the biological therapeutic molecule is not an arginase.

Experimental work with dogs—healthy and those with cancers—carried out by the inventors but also in human patients with terminal hepatocellular carcinoma, have defined the range of insulin infusion rates sufficient to increase the permeability of the vasculature for polypeptides having a molecular weight of 70 kDa or less.

Liver arginase is an enzyme having a molecular weight of about 35 kDa, i.e. a molecular weight that glomerular filtration can remove from plasma. The inventors found that continuous infusion of insulin/glucose resulted in an increase in capillary permeability for arginase sufficient to cause extravasation, thus protecting it from elimination by kidneys.

Albumin has a molecular weight of 72 kDa and usually there is very little loss of it by diffusion into extravascular fluid or by glomerular filtration. The inventors found that continuous infusion of insulin/glucose resulted in a moderate extravasation of albumin causing minor oedema.

Asparaginase in its active form is a tetramer of about 140 kDa molecular weight. The inventors attempted use of insulin/glucose clamp together with asparaginase in its tetrameric form. No evidence for extravasation or glomerular filtration of asparaginase was found when using an insulin-glucose clamp.

Based this experimental work with different proteins distinguished in the molecular weights, the inventors consider it plausible to assume that extravasation of compounds with a molecular weight of 70 Da or less is generally increased by co-administration of an insulin and glucose. This effect can be exploited for improving the delivery and thus the efficacy of biological therapeutic agents.

### The Infusion Device

In most cases, infusions of this kind are carried out in hospitals, under close medical supervision. As the current pandemic of Covid-19 has shown, even in developed countries with sophisticated medical facilities, the capacity for medical care can be brought to the limits. At this stage of the Covid-19 pandemic, a lot of hope is being put into treatments by antibodies and/or antibody fragments. A simple device for controlled infusion rate, which does not require complex and expensive infusion equipment could be of significant help to deliver solutions of antibody fragments and of insulin according to this invention.

Thus, further aspect of the invention is an infusion device for controlled rate of infusion of a liquid medication via an infusion line 4 comprising a fist syringe 2 and a second syringe 6, wherein the first syringe comprises a liquid pharmaceutical composition 1 for administering to a subject by infusion, wherein the pharmaceutical composition comprises at least one pharmaceutical agent, wherein the second syringe comprises a liquid 5, e.g. water or a physiological buffer solution, particularly a liquid without a pharmaceutical agent, and wherein the first syringe 2 is separated from the second syringe 6 by a connector 8 with a pre-set resistance of an orifice 20 to a flow of the liquid 5 from the second syringe 6 to the first syringe 2. Further, the device may comprise a source of pressure, e.g. a source of air pressure for forcing liquid 5 from the syringe 6 into the syringe 2. The source of air pressure may be a further syringe 11, particularly a larger syringe, connected to the second syringe 6, e.g. by a connector 10. In certain embodiments, the further syringe 11 has a volume, which is at least about 5-times, at least about 10.-times or at least bout 15-times and up to about 50-times as high as the volume of the second syringe 6. In certain embodiments, the first syringe 2 and the second syringe 6 are of a loss-of-resistance type.

Still a further aspect of the invention is an infusion kit comprising the syringes 2, 6, and 11, a connector 8 comprising a locking piece 9, a connector 10 comprising a valve, and optionally an infusion line 4.

In particular embodiments, the pharmaceutical agent present in the first syringe 2 is a biological therapeutic agent, particularly an antibody or an antibody fragment, and/or an insulin such as described above.

In particular embodiments, the infusion device is for co-administering a biological therapeutic agent with an insulin-glucose clamp as described above.

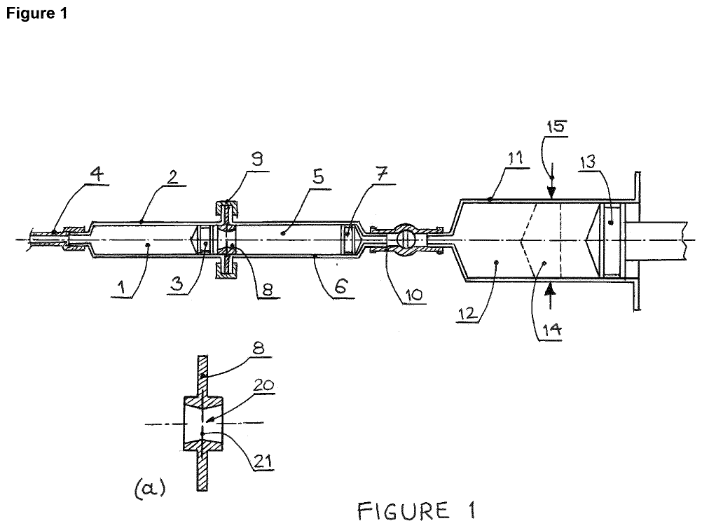

An embodiment of a device is presented in FIG. 1. Medication to be infused (e.g. insulin or a solution with antibody fragments) 1 is filled (or pre-filled) into syringe 2 with a plunger 3. Typically, the size of the syringe 2 is from about 1 to about 10 ml. The syringe 2 is connected back-to-back to syringe 6 filled (or pre-filled) with a liquid 5, e.g. water or a buffer. The plunger 7 is positioned, as shown, at the exit end of the syringe 6. Connecting the two syringes is an orifice connector 8. The locking piece 9 can be separate or integral with the connector 8.

As shown in FIG. 1a, the connector 8 is provided with a fine bore or an orifice 20, which provides a pre-set resistance to the flow rate of liquid, e.g. water from the syringe 6 into syringe 2. To restrict the flow of water to, for example, about 5 ml/h with a driving pressure of 1 bar, the diameter of the orifice 20 is preferably 15 micrometers or less, e.g. about 13 micrometers. Making such small holes can be done in thin metal foil 21, over-molded to make the connector 8.

Different orifices can be provided to control the flow rate of the medication 1 into infusion line 4. The driving pressure to expel water 5 from the syringe 6 into the syringe 2, and thus of the medication 1 into infusion line 4, is provided by air 12, compressed in a large syringe 11. The syringe 11 is connected to the syringe 6 via a connector 10 with a valve. To create a controlled pressure, the plunger 13 of the large syringe may be moved from the starting position to position 14, reducing the volume of the air in the syringe 11 to about a half. It may be locked in that position by clamping as shown by arrow 15, with the air pressure at about 2 bars. If the syringe 11 is of 50 ml volume, and the syringes 2 and 6 are of 5 ml volume, the pressure, and thus the infusion rate would be reduced from the start to the end of infusion by about 20%, which for most practical reasons is acceptable. If needed, the pressure drop can be reduced by a larger volume of syringe 11 or by advancing the plunger 13 past position 14 once or twice during infusion. To minimize effects of friction of the plungers 3 and 7 in the syringes 2 and 6, these syringes should preferably be of loss-of-resistance type.

The orifice for delivering the solution with antibody fragments, or any other protein of interest, could be calibrated to deliver 5 ml in 15 minutes; the one to deliver 5 ml of appropriate insulin solution, could be timed to do so in 60 minutes.

1. An insulin for medical use wherein said insulin is co-administered with glucose and a biological therapeutic molecule.

2. An insulin for the use of claim 1 in human medicine or in veterinary medicine.

3. An insulin for the use of claim 1, wherein said insulin is co-administered with glucose as an insulin-glucose clamp.

4. An insulin for the use of claim 1, wherein the extravasation of the biological therapeutic molecule is assisted.

5. An insulin for the use of claim 1, wherein said insulin is a rapid acting insulin or a short acting insulin, particularly a rapid acting insulin.

6. An insulin for the use of claim 1, wherein said insulin is administered by infusion.

7. An insulin for the use of claim 1, wherein said insulin is administered to a human subject at a rate from 1.5 to 6 I.U./kg body weight//day, preferably from 2 to 4 I.U./kg body weight//day, most preferably about 3 I.U./kg body weight//day.

8. An insulin for the use of claim 1, wherein said glucose is administered by infusion.

9. An insulin for the use of claim 1, wherein said glucose is administered to a subject at a rate to adjust a physiologically acceptable glucose level, e.g. from about 70 mg/dl to about 130 mg/dl for a human subject.

10. An insulin for the use of claim 1, wherein the biological therapeutic molecule is administered by injection or by infusion.

11. An insulin for the use of claim 1, wherein the biological therapeutic molecule has a molecular weight of about 4 kDa to about 70 kDa.

12. An insulin for the use of claim 1, wherein the biological therapeutic molecule is a polypeptide, which is an antibody, an antibody derivative, an antibody fragment, an immunoglobulin fusion protein, an interferon, an interleukin or a cytokine.

13. An insulin for the use of claim 1, wherein the biological therapeutic molecule is a monoclonal antibody fragment.

14. An insulin for the use of claim 12, wherein the biological therapeutic molecule has a molecular weight of about 5 kDa to about 70 kDa, particularly from about 10 kDa to about 50 kDa.

15. An insulin for the use of claim 1, wherein the biological therapeutic molecule is a nucleic acid.

16. An insulin for the use of claim 1, wherein the biological therapeutic molecule is a DNA, an RNA or a modified nucleic acid.

17. An insulin for the use of claim 1, wherein the biological therapeutic molecule is an aptamer, an antisense molecule or a therapeutic RNA.

18. An insulin for the use of claim 1, wherein the biological therapeutic molecule has a molecular weight of about 4 kDa to about 20 kDa, particularly from about 6 kDa to about 10 kDa.

19. An insulin for the use of claim 1, wherein the biological therapeutic molecule is not an asparaginase or an arginase.

20. A method for administering a biological therapeutic molecule to a subject in need thereof, wherein said biological therapeutic molecule is co-administered with an insulin and glucose.

21. The method of claim 20 wherein said subject is a human.

22. The method of claim 20 wherein said subject is a non-human mammal.

23. The method of claim 20 wherein said insulin and said glucose are administered as an insulin-glucose clamp.

24. An infusion device for controlled rate of infusion of a liquid medication 1 via infusion line 4, comprising two interconnected syringes 2 and 6, separated by a connector 8, with a pre-set resistance of an orifice 20 to a flow of liquid 5 from the syringe 6 into the syringe 2.

25. The device according to claim 24, further comprising a source of pressure for forcing liquid 5 from the syringe 6 into the syringe 2.

26. The device according to claim 25 wherein the source of pressure is a source of air pressure.

27. The device according to claim 25 wherein the source of air pressure is a larger syringe 11, connected to syringe 6.

28. The device according to claim 24, where syringes 2 and 6 are of a loss-of-resistance type.

29. An infusion kit comprising the syringes 2, 6, and 11, a connector 8 between syringes 2 and 6 wherein the connector 8 comprises a locking piece 9, a connector 10 between syringes 6 and 11 wherein the connector 10 comprises a valve, and optionally an infusion line 4.
